(19) United States
(12) Patent Application Publication
(10) Pub. No.: US 2002/0058336 A1
(43) Pub. Date: May 16, 2002

(54) Adhesion modulatory peptides and methods for use

(76) Inventor: Samy Ashkar, Boston, MA (US)

Correspondence Address: LAHIVE & COCKFIELD, 28 STATE STREET, BOSTON, MA, 02109, US

(21) Appl. No.: 09/732,411

(22) Filed: Dec. 7, 2000

Related U.S. Application Data
(60) Provisional application No. 60/129,709, filed on Apr. 16, 1999.

(30) Foreign Application Priority Data
Apr. 17, 2000 (US) PCT/US00/10329

(51) Int. Cl. C12N 5/08; C07K 7/04; A61K 38/10; A61K 38/08

(52) U.S. Cl. 435/368; 514/14; 514/16; 514/17; 530/326; 530/328; 530/329

(57) ABSTRACT

The invention provides methods for modulating cell adhesion. The invention also provides novel adhesion modulatory peptides, substrates coated with such adhesion modulatory peptides and devices for modulation of target cell adhesion.

### BACKGROUND OF THE INVENTION

Adhesive interactions are critical in the regulation of multiple physiological and cellular processes including cell proliferation, differentiation, angiogenesis, inflammation, tissue development, cell survival, programmed cell death, and tumor cell growth and metastasis. Moreover in disease, adhesive function is frequently compromised and results in tissue disorder, aberrant cell migration and dysregulation of signaling pathways. Furthermore, regulation of cell adhesive events has broad biomedical implications. For instance, promotion of cell adhesion is desirable, for example, in the seeding of endothelial cells onto vascular grafts, in the stability of medical prostheses, and in promotion of wound healing. Conversely, inhibition of cell adhesion may be of benefit in the treatment of metastasis.

Adhesive events are widely recognized to be important in cell-cell contact as well as in cell interactions between cells and soluble proteins. Moreover, adhesive events are known to involve interactions between substances surrounding the cell (e.g., extracellular matrix molecules, for example fibronectin, vitronectin and laminin) and extracellular adhesion receptors (e.g., integrin receptors). In particular, the integrins are a functionally and structurally related group of receptors that interact with a wide variety of ligands including extracellular matrix glycoproteins, complement and other cells and are involved in many physiologically important processes including hemostasis, thrombosis, wound healing, immune and nonimmune defense mechanisms and oncogenic transformation. Hynes, R. O. (1987) Cell 48:549-554. Several integrins that participate in dynamic cell adhesion bind a tripeptide, arginine-glycine-aspartic acid (RGD), present in their ligand.

Cell adhesion is also mediated by certain adhesive ligands to which extracellular adhesion receptors bind. Among such ligands are the glyco-proteins fibronectin, vitronectin and collagen. All three contain the tripeptide sequence arginine-glycine-aspartic acid (Arg-Gly-Asp or R-G-D) which appears to function as the primary recognition site for receptors on the surface of cells binding to these molecules. Ruoslahti et al. (1987) Science 238:491-497.

### SUMMARY OF THE INVENTION

In view of the importance of promoting cell adhesion or, conversely, for inhibiting adhesion, peptides and compounds suitable for these purposes are desired. In particular, there exists a need for peptides having an amino acid structure that provides the optimum specificity for the receptor of interest.

The present invention satisfies this need and provides related advantages as well. In particular, the present invention features adhesion modulatory peptides which modulate the adhesion of specific cells or cell types based on the adhesion receptors expressed by the specific cell or cell type. The adhesion modulatory peptides are designed to promote and/or enhance the adhesion of specific cells or cell types based on the adhesion receptors expressed by the specific cell or cell type (e.g., the cells receptor expression profile). The present invention features a method of modulating (e.g., enhancing and/or inhibiting) adhesion of a target cell (e.g., endothelial cells, fibroblasts, macrophages, neutrophils and myofiboblasts) to a substrate (e.g., polyvinyl surfaces, gels, collagen, hyaluronic acid, titanium and PGA) which includes providing the cell with an adhesion modulatory peptide-associated substrate such that adhesion of the target cell to the substrate is modulated. The target calls of the present invention can be present in a cell population and/or in a subject (e.g., a human subject).

The present invention also pertains to substrate treated with adhesion modulatory peptides, devices treated with adhesion modulatory peptides and compositions which include the adhesion modulatory peptides of the present invention and a carrier suitable for in vivo use.

### DETAILED DESCRIPTION OF THE INVENTION

The present invention pertains to adhesion modulatory peptides which are designed to promote and/or enhance the adhesion of specific cells or cell types based on the adhesion receptors expressed by the specific cell or cell type (e.g., the cells receptor expression profile). One aspect of the present invention features a method of modulating adhesion of a target cell to a substrate which includes providing the target cell with an adhesion modulatory peptide-associated substrate such that adhesion of the target cell to the substrate is modulated. The language “modulates adhesion” or “modulating adhesion” includes modulating (e.g., stimulating, promoting, enhancing, decreasing or inhibiting) the attachment of a cell to a substrate (e.g., a physical or a molecular substrate). The term “substrate” includes physical materials (e.g., plastic, polyvinyl surfaces, steel, glass, polymers, PGA, metals, for example, titanium) as well as molecular components (e.g., extracellular matrix components, collagen, glycosamoniglycans, for example, hyaluronic acid, chondroitin sulfates and heparan sulfates). Physical materials and/or molecular components can be purified materials or components. Alternatively, physical materials and/or components can be in the form of a composition or biomaterial (e.g., gels). Accordingly, an “adhesion-modulatory peptide” includes a peptide (e.g., at least two amino acid residues joined by a peptide bond or amide bond) which is capable of modulating adhesion or has the ability to modulate (e.g., promote or inhibit) adhesion of a cell to a substrate. Adhesion-modulatory peptides of the invention are at least 3, preferably at least 4, more preferably at least 5, 6, 7, 8, 9, 10, 11, 12, 13, 14, 15, 16, 17, 18, 19, 20, or more amino acid residues in length. Adhesion-modulatory peptides can be between 100 and 2500 Da, between 200 and 2000 Da, between 300 and 1500 Da, or between 500 and 1000 Da. Furthermore, the language “adhesion modulatory peptide-associated substrate” includes a combination or union of an adhesion-modulatory peptide of the present invention in association with a substrate, as defined herein.

The term “target cell includes” a cell (e.g., a mammalian cell) which is capable of binding or has the ability to bind to an adhesion-modulatory peptide or adhesion-modulatory peptide associated substrate of the present invention. In one embodiment, a target cell is present within a subject. In another embodiment, a target cell is isolated from a subject (e.g., a human subject). In yet another embodiment, the target cell is present within a cell population. The term “cell population” includes a collection or group including the target cell and at least a second cell type. Cell populations can also include three, four, five, six or more cell types or can include any number of cell types greater than one (e.g., the target cell and additional undefined cell types). Preferred target cells include, but are not limited to endothelial cells, fibroblasts and macrophages. Additional preferred target cells include, but are not limited to neutrophils and myofiboblasts.

In one embodiment, an adhesion modulatory peptide includes a peptide which specifically enhances adhesion of the target cell to a substrate. The language “specifically enhances” or “specific enhancement” includes a preferred or preferential adhesion (e.g., attachment) of the target cell as compared to a second cell or cell type (e.g., a second cell in a cell population or in a subject). In another embodiment, an adhesion modulatory peptide includes a peptide which specifically inhibits adhesion of the target cell. The language “specifically inhibits” or “specific inhibition” includes a preferential inhibition of adhesion (e.g., attachment) of the target cell as compared to a second cell or cell type (e.g., a second cell in a cell population or in a subject). Preferred adhesion modulatory peptides (e.g., peptides which enhance adhesion) include, but are not limited to, endothelial cell adhesion modulatory peptides, fibroblast adhesion modulatory peptides and macrophage adhesion modulatory peptides. Additional preferred adhesion modulatory peptides (e.g., peptides which inhibit adhesion) include, but are not limited to, neutrophil adhesion modulatory peptides or myofibroblast adhesion modulatory peptides. Particularly preferred adhesion modulatory peptides are set forth in Table II.

In one embodiment, an adhesion modulatory molecule of the present invention enhances binding of an adhesion receptor predominantly expressed by the target cell. The language “predominantly expressed” includes adhesion receptors which are more highly expressed on a cell's surface as compared to other adhesion receptors also expressed on the cell's surface. Preferably, a “predominantly expresses” adhesion receptor is present at least 1.5 times the level of a second adhesion receptor, preferably at least 2 times the level of a second adhesion receptor, more preferably at least 3 times the level of a second receptor, and more preferably at least 5, 10, 50, 100 or 500 times the level of a second receptor. In another embodiment, an adhesion modulatory molecule inhibits binding of an adhesion receptor predominantly expressed by the target cell. The preferential expression of specific adhesion receptors on the surface of a cell (e.g., on the surface of a target cell) provides for preferential or specific adhesion of a preferred cell type (i.e., the target cell).

Yet another aspect on the invention features contacting a substrate with an adhesion modulatory peptide, forming an adhesion modulatory peptide-associated substrate prior to providing the cell with the substrate. The phrase “contacting” includes mixing, incubating or coating the substrate with an adhesion-modulatory peptide of the present invention. For example, the adhesion-modulatory peptides of the present invention can be dissolved or solublized in a suitable solution (e.g., an aqueous solution buffered aqueous solution, organic solvent, buffered organic solvent) and coated on a physical substrate (e.g., a polymer, plastic or biomaterial). Solutions or solvents can be aspirated and/or evaporated to result in a coating of the substrate. Alternatively, the peptides can be covalently adhered to a substrate (e.g., via a covalent modification and/or covalent linkage) or can be adhered via spacer molecules such that steric hindrance of the peptide conformation is diminished or avoided. Spacers, include, but are not limited to aminohexanoic acid, polyglycine, polyamides, polyethylenes, and short functionalized polymers having a carbon backbone (e.g., about 1-12 residues in length). The present invention also pertains to substrate treated with adhesion modulatory peptides, devices (e.g., biomedical devices) treated with adhesion modulatory peptides and compositions which include the adhesion modulatory peptides of the present invention and a carrier suitable for in vivo use.

### Cell Type Specific Expression of Adhesion Receptors

As described above, the adhesion-modulatory peptides of the present invention have particularly utility in the attachment or adhesion of specific cells types (e.g., of a target cell within a cell population). The following section sets forth specific adhesion receptors, in particular, integrins, and describes the naturally-occurring ligands for such adhesion receptors/integrins as well as various cell types which express the particular adhesion receptor/integrin.

Alpha1/beta1 is a receptor for collagen-I collagen-IV and laminin (E1 region). It is expressed on activated T-cells, monocytes, melanoma cells and smooth muscle cells. This integrin is also known as VLA-1 (very late activation antigen 1).

Alpha2/beta1 is a receptor for collagen-I to VI, laminin and possibly fibronectin. It is expressed on B and T lymphocytes, platelets, fibroblasts, endothelial cells and melanoma cells. This receptor is also known as VLA-2 (very late activation antigen 2), GPIa-IIa(glycoprotein Ia-IIa on platelets) and ECMRII (extracellular matrix receptor II)

Alpha3/beta1 is a receptor for epiligrin, laminin (E3 fragment), nidogen/entactin, fibronectin and collagen-1. It is expressed on B-lymphocytes, Kidney glolmerulus and most cultured cell lines. This integrin is also known as VLA-3 (very late activation antigen 3), VCA-2 (very common antigen 2), ECMRI (extracellular matrix receptor I) and Gapb-3 (galactoprotein b3).

Alpha4/beta1 is a receptor for fibronectin containing the CS-1 region, which is situated within the IIICS region, and VCAM-1 (vascular cellular adhesion molecule 1). It is present on lymphocytes, monocytes, eosinophils, NK-cells and thymocytes. This integrin plays a role in the invasion of inflammated tissues, and has also been implicated in skeletal myogenesis, neural crest migration and proliferation, lymphocyte maturation and morphogenisis of the placenta and heart. VCAM-1 is an adhesion molecule which is present on cytokine-activated endothelial cells, while fibronectin is part of the extracellular matrix. Alpha4/beta1 is thus involved in both cell-cell and cell-extracellular matrix adhesion. This integrin is also known as VLA-4 (very late activation antigen 4) and LPAM-2 (lymphocyte Peyer's patch HEV adhesion molecule 2 (mouse)).

Alpha5/beta1 is a receptor for fibronectin. It is expressed on Memory-T-cells, monocytes, platelets and fibroblasts. This integrin is also known as VLA-5 (very late activation antigen 5), FNR (fibronectin receptor), GPIc-IIa (glycoprotein Ic-IIa on platelets) and ECMRVI (extracellular matrix receptor VI).

The alpha4/beta7 integrin is a receptor for MadCAM, fibronectin and VCAM-1. This integrin is only found leukocytes which are directed to the Peyer's Patches of the gut. MadCAM which is an addressin, is only found on Peyer's Patch Endothelium. The alpha4/beta7 integrin is also known as (LPAM-1).

The alpha6/beta1 integrin is expressed on platelets, lymphocytes, monocytes, thymocytes and epithelial cells, on which it functions as a laminin receptor for laminin-1, laminin-2 and laminin-4 in vivo. It is also a receptor for laminin-5, but not in vivo. For laminin-1, the binding site has been localized in the E8 domain of this extracellular matrix molecule. This receptor is also known as VLA-6 (very late activation antigen 6) and GPIc-IIa (glycoprotein Ic-IIa on platelets).

The alpha-6/beta-4 integrin is expressed on different cell-types. It is expressed on immature thymocytes, on squamous epithelia, on subsets of endothelial cells, on Schwann cells and also on fibroblasts in the peripheral nervous system. In stratified epithelia like the skin, alpha-6/beta-4 is concentrated in dense structures which are called hemidesmosomes. These dense structures are involved in the attachment of basal cells to the underlying basement membranes. This is achieved by connection of the intermediate filaments to the extracellular matrix via this integrin. All the other integrins use actin filaments for this purpose in stead of intermediate filaments. The ligands for the alpha-6/beta-4 integrin are laminin-l and laminin-5. The affinity for laminin-5 however is much stronger. In hemidesmosomes it is found attached to laminin-5. The different alpha-6 splice variants do not influence the ligand specificities of the integrin. From studies with knockout mice it was found that in the absence of the integrin (beta-4 knockout or alpha-6 knockout) no hemidesmosomes were present, suggesting that the integrin is necessary for the formation or initiation of hemidesmosomes. these mice showed severe blistering of the skin and died soon after birth.

The alpha7/beta1 integrin is expressed on skeletal and cardiac muscle at specific stages during muscle development. It is a receptor for laminin-1 and binds to it's E8 domain. This integrin is also found localized in focal contacts when melanoma cells attach to laminin-1, while normal melanocytes do not express this integrin. Since alpha7/beta1 is developmentally regulated in muscle cells, it is thought that this integrin has a role in their development. The expression of the three known splice variants is in addition developmentally regulated. Expression of alpha7B precedes the expression of alpha7A and alpha7C. Alpha7/beta1 is also a trophoblast specific laminin receptor on which it may serve a specific function during the early postimplantation period. This integrin is also known as VLA-7 (very late activation antigen 7).

Alpha8/beta1 is a receptor for fibronectin.

AlphaL/beta2 is a receptor for ICAM-1 to 3 (intercellular adhesion molecule 1 to 3). This integrin is only present on leukocytes and plays an important role in interactions between members of this family (e.g. B-cell to T-cell). AlphaL/beta2 is also involved in the interactions between cytotoxic cells and their target-cells. In addition, alphaL/beta2 is crucial for the invasion of leukocytes in tissues. ICAM-1 is expressed on leukocytes and other cells, amongst them are endothelial cells, but only after they have been activated by cytokines for example which are produced in immune reactions and inflammated tissues. ICAM-2 is present on a lot of cells and does not change after cytokine activation. ICAM-3 is primarily expressed on resting lymphocytes and plays a role in the onset of immune reactions. AlphaL/beta2 is normally not activated, but adhesion is induced by activation of the leukocyte, for example by PAF (platelet activating factor) which is produced in inflammated tissues. This integrin is also known as LFA-1 (leukocyte function associated antigen 1).

AlphaM/beta2 is a receptor for C3bi (inactivated form of C3b), factor X (coagulation factor X), fibrinogen and ICAM-1 (intercellular adhesion molecule 1). It is expressed on monocytes, macrophages, NK cells and granulocytes. Alpha-M/beta-2 is important in adherence of monocytes and neutrophils to vascular endothelium, as well as in subsequent extravasation. It also plays a role in phagocytosis of complement coated particles. This integrin is also known as Mac-1 (macrophage receptor 1) and CR-3 (C3bi receptor).

AlphaV/beta1 is a receptor for fibronectin.

AlphaV/beta3 is a receptor for fibrinogen, fibronectin, von Willebrand's factor, Vitronectin, Tsp (Thrombospondin), osteopontin and Bsp1 (bone sialoprotein 1). It is expressed on endothelial cells, some B-cells, platelets and monocytes. alphaVb-beta3 mediates platelet aggregation and endothelial cell adhesion to ECM proteins. This integrin is also known as VNR (vitronectin receptor).

Alphav/beta5 is a receptor for vitronectin. It is expressed on hepatoma cells, fibroblasts and carcinoma cells. This integrin is also known as alphav/betaS and alphav/beta3B.

Alphav/beta6 is a receptor for fibronectin. It is expressed on carcinoma cells.

AlphaX/beta2 is a receptor for fibrinogen. It is found on monocytes, macrophages, granulocytes, NK-cells and activated lymphocytes. This integrin is also known as p150 and CR-4 (C3bi receptor 4).

Alpha-IIb/beta3 is a receptor for fibrinogen, fibronectin, von Willebrand's factor and vitronectin. It is expressed on platelets. This integrin is also known as GPIIb-IIIa (glycoprotein IIIb-IIIa on platelets).

As set forth above, a particular cell type can express predominantly only one or a subset of specific adhesion receptors. An adhesion receptor expression pattern can accordingly be determined for a particular cell type. Given a receptor expression profile for a particular cell in combination with knowledge of the receptor specificity of an adhesion modulatory peptide of the present invention, one can determine additional target cell types on which a particular adhesion modulatory peptide may act or can predict undesireable cross-reactivities (e.g., adhering non-target cells within a cell opulation which inadvertently express the same or a similar receptor expression profile as a target cell).

### Peptides and Peptide Analogs

Table II sets forth preferred adhesion-modulatory peptides of the present invention.

TABLE II

| Peptide | Function |
|---|---|
| SDQDNNGKGSHES | Endothelial cell attachment |
| (SEQ ID NO:1) | |
| SDQDQDGDGHQDS | Endothelial cell attachment |
| (SEQ ID NO:2) | |
| GRGDNPS | Fibronectin receptor binding; |
| (SEQ ID NO:3) | collagenase induction |
| TPVVPTVDTYDGRGDSLAY | β integrin binding |
| (SEQ ID NO:4) | |
| TPVVPTVDTYDGRCD | Cell attachment |
| (SEQ ID NO:5) | |
| DDDRKWGFC | Inhibits collagen interaction |
| (SEQ ID NO:6) | |
| DSVVYGLRSK | Inhibits heparin binding |
| (SEQ ID NO:7) | |
| LDSAS | Inhibits α4 integrin binding |
| (SEQ ID NO:8) | |
| SDV | Inhibits α4 integrin binding |
| HDRKEFAKFEEERARA | β3 attachment |
| (SEQ ID NO:9) | |
| DPGYIGSR | Endothelial cell (EC) attachment; |
| (SEQ ID NO:10) | Competes with SC attachment |
| PNGRCESLAY | Inhibits fibroblast attachment; |
| (SEQ ID NO:11) | Inhibits collagenase induction |
| DRYLKFRPV | Inhibits melanoma cell attachment |
| (SEQ ID NO:12) | |
| KGMNYTVR | neutrophils, endothelium, |
| (SEQ ID NO:13) | fibrosarcomas attachment |
| | melanoma attachment |
| KNNQKSEPLIGRKKT | heparin binding domain |
| (SEQ ID NO:14) | EGF-like motif competes with |
| | binding to glycosaminoglycans |
| | potential anti CD44 (v3vx) activity |
| VLEP | Inhibits VLA-4/VCAM interaction |
| (SEQ ID NO:15) | |

The adhesion-modulatory peptides set forth in table II are described according to standard one-letter amino acid symbols. The following standard abbreviations are also used herein to identify amino acid residues.

TABLE III

| | Three-letter | One-letter |
|---|---|---|
| Amino Acid | Abbreviation | Symbol |
| Alanine | Ala | A |
| Arginine | Arg | R |
| D-Arginine | D-Arg | dR |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The amino acid residues described herein are preferably in the “L” isomeric form. However, residues in the “D” isomeric form can be substituted for any L-amino acid residue, as long as the desired functional property is retained by the peptide. The use of naturally-occurring amino acids may be preferable to other hydrophobic moieties, for example, when coating prosthetic devices to be used in humans, because they are relatively non-immunogenic and non-toxic.

It should be understood that a subject adhesion modulatory peptide need not include a core amino acid residue sequence which is identical to the amino acid residue sequence set forth in Table II, provided that the subject adhesion modulatory peptides retain the ability to specifically bind to a particular adhesion receptor or specifically inhibit binding to a particular adhesion receptor.

Accordingly, an adhesion modulatory peptide of the present invention also includes any analog, fragment or chemical derivative of a peptide whose amino acid residue sequence is shown herein so long as the peptide retains the activity of the adhesion-modulatory peptide from which it is derived. Accordingly, an adhesion modulatory peptide can be subject to various changes, insertions, deletions and substitutions, either conservative or non- conservative, where such changes provide for certain advantages in its use or, at least, are not detrimental to its use.

In this regard, an adhesion modulatory peptide of this invention corresponds to, rather than is identical to, one of the sequence set forth in Table II where one or more changes are made and it retains the ability to specifically bind to a particular adhesion receptor in one or more of the assays as defined herein.

The term “analog” includes any peptide having an amino acid residue sequence substantially identical to a sequence specifically shown herein in which one or more residues have been conservatively substituted with a functionally similar residue and which displays the ability to specifically bind to a particular adhesion receptor or inhibit binding as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The term “analog” also includes any peptide which is structurally similar to an adhesion modulatory peptide of the present invention but which has a chemically derivatized residue(s) and/or peptide linkage in place of a non-derivatized residue or linkage provided that such peptide displays the requisite activity (e.g., functions in a substantially identical manner as the corresponding non-derivatized peptide). “Chemical derivative” refers, for example, to a subject polypeptide having one or more residues and/or linkages chemically derivatized according to routine methodology (e.g., by reaction of a functional side group). The generation of such peptidomimetics may be achieved by techniques of modeling (e.g., computerized molecular modeling) and chemical design known to those of skill in the art.

Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain one or more naturally occurring amino acid derivatives of the twenty standard amino acids. For examples:4-hydroxyproline may be substituted for proline; 5-hydroxylysine may be substituted for lysine; 3-methylhistidine may be substituted for histidine; homoserine may be substituted for serine; and ornithine may be substituted for lysine.

Preferred modification include, for example, modifications designed to enhance chemical stability, increase half-life, increase adsorption (e.g., to an adhesion modulatory peptide-associated substrate), facilitate ease of purification, reduce cost of production, and the like. Particularly preferred modifications are those modifications designed to increase the stability of the adhesion modulatory peptide in vivo. Exemplary modifications are those that block susceptibility to proteolytic activity in biological fluids. Thus a adhesion modulatory peptide can have a stabilizing group at one or both termini. Typical stabilizing groups include amido (e.g., at the C-terminus), acetyl (e.g., at N-terminus), glycerol, benzyl, phenyl, tosyl, alkoxycarbonyl, alkyl carbonyl, benzyloxycarbonyl and the like end group modifications. Additional modifications include using a “L” amino acid in place of a “D” amino acid (e.g., at the termini), cyclization of the polypeptide (e.g., by adding internal cysteine residues capable of forming intramolecular disulfide bridges which cyclize the peptide), and amide rather than amino or carboxy termini to inhibit exopeptidase activity.

Non-hydrolyzable peptide analogs can also be generated using benzodiazepine (see e.g., Freidinger et al. in Peptides: Chemistry and Biology, G. R. Marshall ed., ESCOM Publisher: Leiden, Netherlands, 1988), azepine (see e.g., Huffinan et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J. Med. Chem. 29:295), β-turn dipeptide cores (Nagai et al (1985) Tetrahedron Lett. 26:647; and Sato et al. (1986) J. Chem. Soc. Perkin. Trans. 1:1231), and β-aminoalcohols (Gordon et al. (1985) Biochem. Biophys. Res. Commun. 126:419; and Dann et al. (1986) Biochem. Biophys. Res. Commun. 134:71).

Natural peptide linkages can be replaced by a linkage selected from the group consisting of: —CH₂NH—, —CH₂S—, —CH₂—CH₂—, —CH═CH—(cis and trans), —COCH₂—, —CH(OH)CH₂—, and CH₂SO—, by methods known in the art. A particularly preferred non-peptide linkage is —CH₂NH—.

### Peptide Synthesis

The adhesion modulatory peptides of the present invention can be synthesized by any of the techniques that are known to one of ordinary skill in the art, for example, synthetic chemistry techniques (e.g. solid phase synthesis for solution synthesis) and/or recombinant DNA techniques. Synthetic chemistry techniques (e.g. solid phase synthesis) may be preferred for reasons of purity, freedom from undesired side products, and ease of product purification.

Briefly, the solid phase synthesis methods include the sequential addition of one or more amino acid residues or protected amino acid residues to a growing peptide chain. Normally, either the amino or carboxyl group of the first amino residue is protected by a suitable protecting group. A different protecting group can be used for amino acids containing a reactive side chain (e.g. lysine).

As a general first step, a protected first amino acid residue is attached to an inert solid support through its unprotected carboxyl or amino group. The protecting group is then removed and a second amino acid residue in the sequence (suitably protected) is admixed and reacted under conditions suitable for forming an amide linkage with the first amino acid residue with the first amino acid residue attached to the solid support. The protecting group of the second amino acid residue is then removed and a third amino acid residue (likewise protected) is added, and so forth. After all of the desired amino acids have been linked in the proper sequence, any remaining terminal and side group protecting groups (and solid support) are removed sequentially or concurrently, to afford the final peptide. Preferably, the linear sequence is synthesized using commercially available automated peptide synthesizers. The material so synthesized can be precipitated and further purified, for example by high performance liquid chromatography (HPLC). Although a purity of greater than 95 percent for the synthesized peptide is preferred, lower purity may be acceptable.

Alternatively, the peptides of the present invention can be produced by recombinant DNA techniques in a host cell transformed with a nucleic acid having a sequence encoding such peptide. To produce a peptide by recombinant techniques, host cells (e.g., bacterial cells such as E. coli, insect cells, yeast, or mammalian cells, for example, Chinese hamster ovary (CHO) cells) are transformed with a vector suitable for expressing a peptide of the invention and cultured in a medium such that the cells produce the peptides. Peptides so-produced can be purified from cell culture medium, host cells, or both using techniques known in the art for purifying peptides including ultrafiltration, ion-exchange chromatography, gel filtration chromatography, electrophoresis or immunopurification with antibodies specific for the peptide.

Accordingly, the present invention provides nucleic acid molecules which encode the peptides of the present invention, expression vectors and host cells suitable for expression of such peptides. Nucleic acid coding for the peptides of the invention can easily be synthesized by chemical techniques, for example, the phosphotriester method of Matteucci et al., J. Am. Chem. Soc., 103:3185 (1981). Moreover, by chemically synthesizing the coding sequence, modifications can be made by substituting the appropriate bases for those encoding the native amino acid residue sequence.

Suitable expression vectors, promoters, enhancers, and other expression control elements may be found in Sambrook et al Molecular Cloning: A Laboratory Manual, second edition, Cold Spring Harbor Laboratory Press, Cold Spring Harbor, N.Y., 1989. Other suitable expression vectors, promoters, enhancers, and other expression elements are known to those skilled in the art. Expression in mammalian, yeast or insect cells leads to partial or complete glycosylation of the recombinant material and formation of any inter- or intra-chain disulfide bonds. Suitable vectors for expression in yeast include YepSec1 (Baldari et al. (1987) Embo J. 6: 229-234); pMFa (Kurjan and Herskowitz (1982) Cell 30: 933-943); JRY88 (Schultz et al. (1987) Gene 54: 113-123) and pYES2 (Invitrogen Corporation, San Diego, Calif.). These vectors are freely available. Baculovirus and mammalian expression systems are also available. For example, a baculovirus system is commercially available (PharMingen, San Diego, Calif.) for expression in insect cells while the pMSG vector is commercially available (Pharmacia, Piscataway, N.J.) for expression in mammalian cells.

For expression in E. coli, suitable expression vectors include, among others, pTRC (Amann et al. (1988) Gene 69: 301-315); pGEX (Amrad Corp., Melbourne, Australia); pMAL (N. E. Biolabs, Beverly, Mass.); pRIT5 (Pharmacia, Piscataway, N.J.); pET-11 d (Novagen, Madison, Wis.) Jameel et al., (1990) J. Virol 64:3963-3966; and pSEM (Knapp et al. (1990) BioTechniques 8: 280-281). The use of pTRC, and pET-11 d, for example, will lead to the expression of unfused protein. The use of pMAL, pRIT5 pSEM and pGEX will lead to the expression of peptide fused to maltose E binding protein (pMAL), protein A (pRIT5), truncated β-galactosidase (PSEM), or glutathione S-transferase (pGEX). When a peptide of the invention is expressed as a fusion protein, it is particularly advantageous to introduce an enzymatic cleavage site at the fusion junction between the carrier protein and the peptide. The peptide may then be recovered from the fusion protein through enzymatic cleavage at the enzymatic site and biochemical purification using conventional techniques for purification of proteins and peptides. Suitable enzymatic cleavage sites include those for blood clotting Factor Xa or thrombin for which the appropriate enzymes and protocols for cleavage are commercially available from, for example, Sigma Chemical Company, St. Louis, Mo. and N.E. Biolabs, Beverly, Mass. The different vectors also have different promoter regions allowing constitutive or inducible expression with, for example, IPTG induction (PRTC, Amann et al., (1988) supra; pET-11 d, Novagen, Madison, Wis.) or temperature induction (pRIT5, Pharmacia, Piscataway, N.J.). It may also be appropriate to express recombinant peptides in different E. coli hosts that have an altered capacity to degrade recombinantly expressed proteins (e.g. U.S. Pat. No. 4,758,512). Alternatively, it may be advantageous to alter the nucleic acid sequence to use codons preferentially utilized by E. coli, where such nucleic acid alteration would not affect the amino acid sequence of the expressed peptide.

Host cells can be transformed to express the nucleic acid sequences of the invention using conventional techniques such as calcium phosphate or calcium chloride co-precipitation, DEAE-dextran-mediated transfection, or electroporation. Suitable methods for transforming the host cells may be found in Sambrook et al. supra, and other laboratory textbooks. The nucleic acid sequences of the invention may also be chemically synthesized using standard techniques (i.e. solid phase synthesis).

The present invention also provides nucleic acid sequences encoding peptides of the invention. Nucleic acid sequences used in any embodiment of this invention can be cDNA obtained from cDNAs encoding the corresponding peptide sequences, or alternatively, can be any oligodeoxynucleotide sequence having all or a portion of a sequence represented herein, or their functional equivalents. Such oligodeoxynucleotide sequences can be produced chemically or mechanically, using known techniques. A functional equivalent of an oligonucleotide sequence is one which is 1) a sequence capable of hybridizing to a complementary oligonucleotide to which the sequence (or corresponding sequence portions) of the peptide, or fragments thereof, hybridizes, or 2) the sequence (or corresponding sequence portion) complementary to the nucleic acid sequences encoding the peptide sequence. Whether a functional equivalent must meet one or both criteria will depend on its use.

The present invention also provides a method of producing isolated adhesion modulatory peptides of the invention or portions thereof comprising the steps of culturing a host cell transformed with a nucleic acid sequence encoding an adhesion modulatory peptide of the invention in an appropriate medium to produce a mixture of cells and medium containing said adhesion modulatory peptide; and purifying the mixture to produce substantially pure adhesion modulatory peptide. Host cells transformed with an expression vector containing DNA coding for an adhesion modulatory peptide of the invention or a portion thereof are cultured in a suitable medium for the host cell. Adhesion modulatory peptides of the invention can be purified from cell culture medium, host cells, or both using techniques known in the art for purifying peptides and proteins including ion-exchange chromatography, gel filtration chromatography, ultrafiltration, electrophoresis and immunopurification with antibodies specific for the adhesion modulatory peptides or portions thereof of the invention.

### Therapeutic Uses

The adhesion modulatory peptides of the present invention were identified according to functional screening assays (e.g., assays designed to screen test peptides for their ability to perform a desired biological function). Accordingly, the adhesion modulatory peptides of the present invention have general utility in promoting the adhesion of cells to, for example, physical substrates, molecular substrates, biomaterials (e.g., reconstructive biomaterials) and prosthetic devices. In particular, the adhesion modulatory peptides of the present invention have the following utilities.

The adhesion modulatory peptides of the present invention (e.g., endothelial cell adhesion modulatory peptides) have particular utility in promoting attachment of endothelial cells and further promote endothelial cell retention and spreading. Accordingly, the endothelial cell adhesion modulatory peptides of the present invention have utility in improving endothelial cell adhesion to vascular graft surfaces. For instance, it is known that vascular grafts do not spontaneously endothelialize in humans. Accordingly, treatment of graft surfaces and/or synthetic graft materials (e.g., polytetrafluoroethylene (“ePTFE”) or polyethylene terephthalate) with endothelial cell adhesion modulatory peptide prior to grafting can increase endothelial cell attachment as well as endothelial cell retention and spreading. Accordingly, the endothelial cell adhesion modulatory peptides of the present invention can be used in the regulation of vessel growth during wound healing and/or in the treatment of damage resulting from vascular disease.

The adhesion modulatory peptides of the present invention have further utility in inhibiting or preventing cellular apoptosis. For example, it is known that certain cells are dependent on adhesion or adherence to a substrate for survival (e.g., adhesion-dependent cells). Accordingly, adhesion-enhancing peptides of the present invention can be used, for example, to prevent or inhibit cells form apoptosis (e.g., rescue cells from matrix-induced programmed cell death) by providing cell-substrate contacts. Alternatively, the adhesion-inhibitory peptides of the present invention can be used to induce apoptosis in adhesion-dependent cells.

The adhesion-modulatory peptides of the present invention further have utility in tissue engineering. Tissue engineering techniques involve culturing a variety of tissues both in vitro and in vivo using polymer “scaffolds” (e.g., scaffolds made of biomaterials, for example, biodegradable materials) to support tissue growth. The adhesion modulatory peptides of the present invention can be used to stimulate and/or enhance cell attachment to such polymer scaffolds and concomitantly enhance tissue growth. Moreover, use of the cell adhesion modulatory peptides of the present invention to modify the surfaces of synthetic materials used in medical implants results in faster and more complete tissue integration as well as a reduction in foreign body response.

The peptides of the present invention may be utilized for many in vivo medical uses such as coating of medical devices, including prostheses or implants, for example vascular implants, so as to facilitate the attachment of cells thereto.

Moreover, the adhesion-modulatory molecules of the present invention have specific activities which are based, at least in part, on their ability to bind or preferentially bind a specific adhesion receptor or particular cell type. Such specific activities are set forth below.

Endothelial cell-specific adhesion peptides, also referred to as “endothelial cell attachment peptides” interact, in particular, with an EGF-like domain on an endothelial cell. For example, endothelial cell-specific adhesion peptides bind cells via heparan sulfate or ICAM molecules on the endothelial cell surface, as compared to binding the cell via an integrin. Such endothelial cell-specific adhesion peptides include, for example, SDQDNNGKGSHES (SEQ ID NO:1) and SDQDQDGDGHQDS (SEQ ID NO:2).

Fibronectin receptor-binding peptides, for example, GRGDNPS (SEQ ID NO:3), ligate integrin receptors on the cell surface, upregulate metalloproteases (e.g., collagenase I) which are specific to remodelling systems (as contrasted with general destructive enzymes). Accordingly, such peptides are useful in the treatment of fibrosis (e.g., chondrofibrosis), in particular, in the clearing of debris.

Adhesion-modulatory peptides, for example, the peptide TPVVPTVDTYDGRGD (SEQ ID NO:5) are specific for αvβ3 integrin expressing cells (e.g., activated macrophages, and regenerating endothelial cells) and, in particular, have utility during vascularization.

Adhesion-modulatory peptides, for example, DDDRKWGFC (SEQ ID NO:6) inhibit cell binding to collagen (via the β1 subunit of integrins). During wound healing, recruited fibroblasts transiently differentiate into myofibroblasts, which express α2β1 integrins, (the α2 integrin subunit being responsible for actin binding). Accordingly, myofibroblasts bind both collagen at the wound site and actin, resulting in wound contraction, as well as wrinkling and scarrring. Accordingly, peptides which specifically inhibit at least the collagen binding of such cells, can minimize wound contraction, resulting in reduced kelloid tissue formation and scarring.

Adhesion-modulatory peptides, for example DSVVYGLRSK (SEQ ID NO:6) inhibit the binding of heparin to proteins or cell binding to glycosaminoglycans and accordingly, have potential utility as anticlotting agents.

Adhesion-modulatory peptides, for example, LDSAS (SEQ ID NO:8) and SDV specifically inhibit α4 integrin binding. α4 has been demonstrated to be important, for example, in cell migration through vessels. Accordingly, such adhesion modulatory peptides may have immunomodulatory effects and/or anti-cancer effects.

Adhesion-modulatory peptides, for example, DPGYIGSR (SEQ ID NO:10), inhibit endothelial cell attachment, in particular by competing for βVβ3 integrin binding on the cell surface. Accordingly, such peptides may have utility as anti-angiogenic factors.

Adhesion-modulatory peptides, for example, KNNQKSEPLIGRKKT (SEQ ID NO:14) include an EGF-like motif which specifically competes with binding of cells to glycosaminoglycans. In particular, such peptides have anti-CD44 (v3vx) activity and have potential use as anti-tumorigenic agents.

Adhesion-modulatory peptides, for example, DRYLKFRPV (SEQ ID NO:12) specifically inhibit melanoma cell attachment. Melanoma cells, in particular, have a distinct receptor expression profile (e.g., a low integrin variable profile). Accordingly, such peptides can be used to sequester melanoma cells, by forming a physical barrier of peptide-associated substrate around a melanoma, thereby preventing its metastasis.

Adhesion-modulatory peptides, for example, PNGRGESLAY (SEQ ID NO:11) can function as RGD analogs (e.g., by binding integrins), and accordingly may have antithritic activity (e.g., a dis-integrin activity).

Adhesion-modulatory molecules, for example, KGMNYTVR (SEQ ID NO:13) adhere neutrophils and accordingly may have an anti-bacterial or bacteriocidal effect.

### Therapeutic Compositions and Preparations

The present invention provides therapeutic compositions comprising isolated peptides or analogs thereof and a pharmaceutically acceptable carrier, or diluent. Administration of the therapeutic compositions of the present invention to an individual can be carried out using known techniques. Peptides or analogs thereof may be administered to an individual in combination with, for example, an appropriate diluent, adjuvant and/or a carrier. Pharmaceutically acceptable diluents include saline and aqueous buffer solutions. Pharmaceutically acceptable carriers include polyethylene glycol (Wie et al. (1981) Int. Arch. Allergy Appl. Immunol. 64:84-99) and liposomes (Strejan et al. (1984) J. Neuroimmunol 7: 27). The carrier can also include a matrix, e.g., fibrin, collagen, gelatin, agarose, calcium phosphate containing compounds and combinations thereof. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether.

Administration of the therapeutic compositions of the present invention to an individual can be carried out using known procedures at dosages and for periods of time effective to significantly reduce or eliminate symptoms associated with the condition or disease being treated. Effective amounts of the therapeutic compositions will vary according to the age, sex, and weight of the “subject”, and the ability of the peptide to perform its intended function. The term “subject” is intended to include subjects susceptible to the particular condition or disease being treated. The term “subject” is intended to include mammals, particularly humans.

In addition to compositions containing a single peptide, mixtures of at least two peptides (i.e., a physical mixture of at least two peptides) can also be provided. Such compositions can be administered in the form of a therapeutic composition with a pharmaceutically acceptable carrier or diluent. A therapeutically effective amount of one or more of such compositions can be administered simultaneously or sequentially. Preferred therapeutic compositions comprise peptides which include the peptides having the amino acid sequences shown in SEQ ID NOs:1-15. Dosage regima may be adjusted to provide the optimum therapeutic response. For example, several divided doses may be administered daily or the dose may be proportionally reduced as indicated by the exigencies of the therapeutic situation. A therapeutically effective amount is that amount sufficient to significantly reduce or alleviate symptoms associated with the particular condition or disease being treated. A preferred composition of the present invention is a wound healing composition. The wound healing composition contains a wound healing effective amount of adhesion-modulatory peptide of the invention.

The peptide or analog thereof may be administered in a convenient manner such as by injection (subcutaneous, intravenous, etc.), oral administration, inhalation, transdermal application, or rectal administration. Depending on the route of administration, the active compound may be coated within a material to protect the compound from the action of enzymes, acids and other natural conditions which may inactivate the compound.

To administer a peptide by other than parenteral administration, it may be necessary to coat the peptide with, or co-administer the peptide with, a material to prevent its inactivation. For example, peptide may be co-administered with enzyme inhibitors or in liposomes. Enzyme inhibitors include pancreatic trypsin inhibitor, diisopropylfluorophosphate (DEP) and trasylol. Liposomes include water-in-oil-in-water CGF emulsions as well as conventional liposomes (Strejan et al., (1984) J. Neuroimmunol. 7:27).

The peptide or analog may also be administered parenterally or intraperitoneally. Dispersions can also be prepared in glycerol, liquid polyethyline glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions of dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glyceral, propylene glycol, and liquid polyetheylene glycol, and the like), suitable mixtures thereof, and vegetable oils. The proper fluidity can be maintained, for example, by the use of a coating such as licithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thirmerosal, and the like. In many cases, it will be preferable to include isotonic agents, for example, sugars, polyalcohols such as manitol and sorbitol or sodium chloride in the composition. Prolonged absorption of the injectable compositions can be brought about, including in the composition, an agent which delays absorption, for example, aluminum monostearate and gelatin.

Sterile injectable solutions can be prepared by incorporating active compound (i.e., peptide or fragment thereof) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., peptide or fragment thereof) plus any additional desired ingredient from a previously sterile-filtered solution thereof

When a peptide of the invention is suitably protected, as described above, the peptide may be orally administered, for example, with an inert diluent or an assimilable edible carrier. The peptide and other ingredients may also be enclosed in a hard or soft shell gelatin capsule, compressed into tablets, or incorporated directly into the individual's diet. For oral therapeutic administration, the peptide may be incorporated with excipients and used in the form of ingestible tablets, buccal tablets, troches, capsules, elixirs, suspensions, syrups, wafers, and the like. Such compositions and preparations should contain at least 1% by weight of peptide. The percentage of the composition and preparations may, of course, be varied and may conveniently be between about 5 to 80% of the weight of the unit. The amount of peptide in such therapeutically useful compositions is such that a suitable dosage will be obtained.

The tablets, troches, pills, capsules and the like may also contain the following: a binder such as gum gragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservative, a dye and flavoring such as cherry or orange flavor. Of course, any material used in preparing any dosage unit form should be pharmaceutically pure and substantially non-toxic in the amounts employed. In addition, the peptide or analog may be incorporated into sustained-release preparations and formulations.

The peptide or analog may also be administered topically. The use of a non-aqueous lipid miscible carrier, for example, such as prepared with liposomes are particularly advantageous since they provide improved activity at the treatment site (e.g., the wound site).

The language “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. The language “dosage unit form” includes physically discrete units suited as unitary dosages for the mammalian subjects to be treated; each unit containing a predetermined quantity of active compound calculated to produce the desired therapeutic effect in association with the required pharmaceutical carrier. The specification for the novel dosage unit forms of the invention are dictated by and directly dependent on (a) the unique characteristics of the peptide or analog and the particular therapeutic effect to be achieved, and (b) the limitations inherent in the art of compounding such a peptide or analog for the treatment of sensitivity in individuals.

Appropriate dosages of the peptides of the invention will depend upon the condition presented by the individual subject. The skilled medical worker will be able to determine appropriate dosages required to combat the physiological activity. However, in general, amounts of from about 1 μg to 100 μg/kg body weight/day of the biologically active peptide should be useful.

The entire contents of all of the references (including literature references, issued patents, and published patent applications) cited throughout this application are hereby expressly incorporated by reference.

We claim:

1. A method of modulating adhesion of a target cell to a substrate, comprising providing the target cell with an adhesion modulatory peptide-associated substrate such that adhesion of the target cell to the substrate is modulated.

2. The method of claim 1, wherein the adhesion modulatory peptide comprises a peptide which specifically enhances adhesion of the target cell.

3. The method of claim 1, wherein the adhesion modulatory peptide comprises a peptide which specifically inhibits adhesion of the target cell

4. The method of claim 1, wherein the adhesion modulatory peptide is selected from the group consisting of an endothelial cell adhesion modulatory peptide, a fibroblast adhesion modulatory peptide and a macrophage adhesion modulatory peptide.

5. The method of claim 4, wherein the adhesion modulatory peptide is an endothelial cell adhesion modulatory peptide.

6. The method of claim 4, wherein the adhesion modulatory peptide is a fibroblast adhesion modulatory peptide.

7. The method of claim 4, wherein the adhesion modulatory peptide is a neotrophil adhesion modulatory peptide or a myofibroblast adhesion modulatory peptide.

8. The method of claim 1, wherein the adhesion modulatory peptide comprises an amino acid residue sequence selected from the group consisting of SDQDNNGKGSHES (SEQ ID NO:1), SDQDQDGDGHQDS (SEQ ID NO:2), GRGDNPS (SEQ ID NO:3), TPVVPTVDTYDGRGDSLAY (SEQ ID NO:4), TPVVPTVDTYDGRGD (SEQ ID NO:5), HDRKEFAKFEEERARA (SEQ ID NO:10), DPGYIGSR (SEQ ID NO:10), KGMNYTVR (SEQ ID NO:13), and VLEP (SEQ ID NO:15).

9. The method of claim 1, wherein the adhesion modulatory peptide comprises an amino acid residue sequence selected from the group consisting of DDDRKWGFC (SEQ ID NO:6), DSVVYGLRSK (SEQ ID NO:7), LDSAS (SEQ ID NO:8), SDV (SEQ ID NO:9), PNGRGESLAY (SEQ ID NO:11), and DRYLKFRPV (SEQ ID NO:12).

10. The method of claim 1, wherein the adhesion modulatory molecule enhances binding of an adhesion receptor predominantly expressed by the target cell.

11. The method of claim 1, wherein the adhesion modulatory molecule inhibits binding of an adhesion receptor predominantly expressed by the target cell.

12. The method of claim 1, wherein the target cell is selected from the group consisting of an endothelial cell, a fibroblast and a macrophage.

13. The method of claim 12, wherein the target cell is an endothelial cell.

14. The method of claim 12, wherein the target cell is a fibroblast.

15. The method of claim 1, wherein the target cell is a neutrophil or a myofiboblast.

16. The method of claim 1, wherein the target cell is within a cell population.

17. The method of claim 1, wherein the target cell is within a subject.

18. The method of claim 1, wherein the substrate is selected from the group consisting of a polyvinyl surface, a gel, collagen, hyaluronic acid, titanium and PGA.

19. The method of claim 1, further comprising contacting the substrate with the adhesion modulatory peptide, forming the adhesion modulatory peptide-associated substrate prior to providing the cell with the substrate.

20. An adhesion modulatory peptide which modulates adhesion of a target cell to a substrate.

21. The adhesion modulatory peptide of claim 20, wherein the peptide enhances adhesion of a target cell to a substrate.

22. The adhesion modulatory peptide of claim 20, wherein the peptide inhibits adhesion of a target cell to a substrate.

23. The adhesion modulatory peptide of claim 20, comprising an amino acid residue sequence selected from the group consisting of SDQDNNGKGSHES (SEQ ID NO:1), SDQDQDGDGHQDS (SEQ ID NO:2), GRGDNPS (SEQ ID NO:3), TPVVPTVDTYDGRGDSLAY (SEQ ID NO:4), TPVVPTVDTYDGRGD (SEQ ID NO:5), HDRKEFAKFEEERARA (SEQ ID NO:9), DPGYIGSR (SEQ ID NO:10), KGMNYTVR (SEQ ID NO:13) and VLEP (SEQ ID NO:15).

24. The adhesion modulatory peptide of claim 20, comprising an amino acid residue sequence selected from the group consisting of DDDRKWGFC (SEQ ID NO:6), DSVVYGLRSK (SEQ ID NO:7), LDSAS (SEQ ID NO:8), SDV (SEQ ID NO:9), PNGRGESLAY (SEQ ID NO:12), and DRYLKFRPV (SEQ ID NO:13).

25. The adhesion modulatory peptide of claim 20 having a molecular weight less than about 2500 Da.

26. A substrate treated with the adhesion modulatory peptide of claim 20.

27. A device treated with the adhesion modulatory peptide of claim 20.

28. A composition comprising the adhesion modulatory peptide of claim 20 and a carrier suitable for in vivo use.

29. A device for modulation of adhesion of a target cell comprising a substrate in combination with an adhesion-modulatory peptide, forming a device for modulating adhesion.


SEQUENCE LISTING (15 sequences)

SEQ ID NO: 1 - protein, 13 aa, Artificial Sequence
  Description of Artificial SequenceSynthetic peptide
SDQDNNGKGSHES

SEQ ID NO: 2 - protein, 13 aa, Artificial Sequence
  Description of Artificial SequenceSynthetic peptide
SDQDQDGDGHQDS

SEQ ID NO: 3 - protein, 7 aa, Artificial Sequence
  Description of Artificial SequenceSynthetic peptide
GRGDNPS

SEQ ID NO: 4 - protein, 19 aa, Artificial Sequence
  Description of Artificial SequenceSynthetic peptide
TPVVPTVDTYDGRGDSLAY

SEQ ID NO: 5 - protein, 15 aa, Artificial Sequence
  Description of Artificial SequenceSynthetic peptide
TPVVPTVDTYDGRGD

SEQ ID NO: 6 - protein, 9 aa, Artificial Sequence
  Description of Artificial SequenceSynthetic peptide
DDDRKWGFC

SEQ ID NO: 7 - protein, 10 aa, Artificial Sequence
  Description of Artificial SequenceSynthetic peptide
DSVVYGLRSK

SEQ ID NO: 8 - protein, 5 aa, Artificial Sequence
  Description of Artificial SequenceSynthetic peptide
LDSAS

SEQ ID NO: 9 - protein, 16 aa, Artificial Sequence
  Description of Artificial SequenceSynthetic peptide
HDRKEFAKFEEERARA

SEQ ID NO: 10 - protein, 8 aa, Artificial Sequence
  Description of Artificial SequenceSynthetic peptide
DPGYIGSR

SEQ ID NO: 11 - protein, 10 aa, Artificial Sequence
  Description of Artificial SequenceSynthetic peptide
PNGRGESLAY

SEQ ID NO: 12 - protein, 9 aa, Artificial Sequence
  Description of Artificial SequenceSynthetic peptide
DRYLKFRPV

SEQ ID NO: 13 - protein, 8 aa, Artificial Sequence
  Description of Artificial SequenceSynthetic peptide
KGMNYTVR

SEQ ID NO: 14 - protein, 15 aa, Artificial Sequence
  Description of Artificial SequenceSynthetic peptide
KNNQKSEPLIGRKKT

SEQ ID NO: 15 - protein, 4 aa, Artificial Sequence
  Description of Artificial SequenceSynthetic peptide
VLEP